(19) United States
(12) Patent Application Publication
(10) Pub. No.: US 2009/0170133 A1
(43) Pub. Date: Jul. 2, 2009

(54) Intact IGFBP-3 as a Colon Cancer Risk Factor in Patients With Inflammatory Bowel Disease

(76) Inventors: Irena Kirman, Fort Lee, NJ (US); Richard L. Whelan, New York, NY (US)

Correspondence Address: COOPER & DUNHAM, LLP, 30 Rockefeller Plaza, 20th Floor, NEW YORK, NY, 10112, US

(21) Appl. No.: 11/918,337

(22) PCT Filed: Apr. 19, 2006
(86) PCT No.: PCT/US2006/014912

§ 371 Date: Feb. 24, 2009

Related U.S. Application Data
(60) Provisional application No. 60/673,192, filed on Apr. 19, 2005.

(51) Int. Cl. G01N 33/53

(52) U.S. Cl. 435/7.92; 435/7.1

(57) ABSTRACT

The subject invention provides a method for determining whether a human subject afflicted with long-lasting irritable bowel disease (IBD) has an increased risk for developing colon cancer comprising: (a) determining the concentration of intact IGFBP-3 in a suitable cell-free bodily fluid sample taken from the subject; and (b) determining whether the concentration of intact IGFBP-3 determined in step (a) is indicative of an increased risk of colon cancer in a human subject afflicted with long-lasting IBD. This invention also provides a kit for performing the instant method.

This application claims the benefit of U.S. Provisional Application No. 60/673,192, filed Apr. 19, 2005, the contents of which are incorporated hereby by reference into the subject application.

Throughout this application, various publications are referenced by number. Full citations for these publications may be found at the end of the specification immediately preceding the claims. The disclosures of these publications are hereby incorporated by reference into this application to describe more fully the art to which this invention pertains.

### BACKGROUND OF THE INVENTION

Epithelial cell growth regulation has been reported to be altered in inflammatory bowel disease (IBD) patients. The cell growth regulatory factor, insulin-like growth factor biding protein 3 (IGFBP-3), may be partly responsible for this phenomenon. Thus far, IGFBP-3 levels have been assessed as values of total protein, which is a sum of bioactive intact 43-45 kDa protein and its inactive proteolytic cleavage fragments.

Inflammatory bowel disease (IBD), which includes Crohn's disease (CD) and ulcerative colitis (UC), is a chronic inflammatory intestinal disorder of unknown etiology. An altered pattern of cell growth has been noted in IBD patients; in several studies the rate of epithelial cell proliferation has been reported to be increased (1,2), especially in the areas of dysplasia (3). Further, an increased rate of apoptosis has been demonstrated in epithelial cells from IBD patients (4-6). These observations suggest that bioactive proteins that regulate cell growth, namely the growth factors, could be involved in accelerated cell cycling. Several studies have investigated the serum concentration of IGF-I in both children and adults suffering from IBD and have found serum levels to be significantly decreased (7-9). Thus, a deficiency in IGF-I, a major cell growth stimulator during an IBD relapse, may account, at least in part, for an elevated rate of intestinal epithelial cell apoptosis. It is not clear, however, what factors, if any, are responsible for the increased rate of epithelial cell proliferation.

The growth stimulatory effects of IGF-I are regulated by its binding protein, IGFBP-3. IGFBP-3, originally described as IGF-I and IGF-II binding protein, has since been shown to have an independent cell growth regulatory effect. After binding to the cell surface, IGFBP-3 is rapidly internalized and translocated to the nucleus (10-12). IGFBP-3-related cell growth regulatory effects might be linked to its ability to modulate the expression of survival versus apoptosis-related genes (13). IGFBP-3 has been shown to induce apoptosis and to inhibit proliferation of human breast, prostate, lung and colon cancer cells (14-17). In addition, IGFBP-3 has also been shown to inhibit growth of other types of cells, both normal and transformed. These findings suggest that changes in IGFBP-3 concentration may lead to alterations in cell growth regulation. Several studies have reported that plasma IGFBP-3 levels are decreased during episodes of active IBD whereas during remissions, levels are in the normal range (7-9). These studies used immunoassays which determine total IGFBP-3 concentrations. The total concentration is the sum of both the intact bioactive IGFBP-3 and the inactive cleavage products.

### SUMMARY OF THE INVENTION

The subject invention provides a method for determining whether a human subject afflicted with long-lasting irritable bowel disease (IBD) has an increased risk for developing colon cancer comprising:
(a) determining the concentration of intact IGFBP-3 in a suitable cell-free bodily fluid sample taken from the subject; and
(b) determining whether the concentration of intact IGFBP-3 determined in step (a) is indicative of an increased risk of colon cancer in a human subject afflicted with long-lasting IBD.

This invention also provides a kit for determining whether a human subject afflicted with long-lasting irritable bowel disease (IBD) has an increased risk for developing colon cancer comprising, in separate compartments:
(a) an ELISA plate having operably affixed thereto anti-IGFBP-3 antibodies, wherein collectively the antibodies bind to both intact and degraded IGFBP-3;
(b) magnetic beads having operably affixed thereto anti-IGFBP-3 antibodies, wherein collectively the antibodies bind to both intact and degraded IGFBP-3; and
(c) a detectably-labeled antibody which binds to both intact and degraded IGFBP-3, wherein the detectably-labeled antibody permits the quantification of total IGFBP-3 bound to the ELISA plate of (a).

### BRIEF DESCRIPTION OF THE FIGURES

FIG. 1. The Concentration of Intact and Total IGFBP-3 is Equally Decreased in Patients with Active Moderate-to Severe IBD. Total IGFBP-3 concentration was measured in plasma from IBD patients in ELISA. The level of intact IGFBP-3 was assessed in a combined ELISA and Western Blot assay. Data are shown as medians and upper quartile values. ***p<0.005 compared to remission and controls; *p<0.05 compared to remission and controls; ̂ p<0.05 versus remission; ° p<0.05 compared to controls only.

FIG. 2. A Significant Depletion of Intact IGFBP-3 Occurs in a Certain Proportion of IBD Patients. Western blot analysis of IGFBP-3 has been performed in plasma samples from IBD patients as described in Materials and Methods. Depletion of intact IGFBP-3 can be seen in lanes 6 and 9.

FIG. 3. Proteases in Plasma from IBD Patients. Plasma proteolytic activity has been analyzed in zymography as described in Materials and Methods. Panel A. The most abundant protease in plasma samples (lanes 1-10) correspond to a 92 kDa pro-form of MMP-9. A dimeric pro-form of MMP-9 can be seen as a 225 kDa protease. A 72 kDa protease corresponds to MMP-2. Panel B shows a Western Blot analysis confirming that a 92 kDa protease is indeed MMP-9.

### DETAILED DESCRIPTION OF THE INVENTION

### Terms

“Antibody” shall include, without limitation, (a) an immunoglobulin molecule comprising two heavy chains and two light chains and which recognizes an antigen; (b) polyclonal and monoclonal immunoglobulin molecules; and (c) monovalent and divalent fragments thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. Antibodies can be both naturally occurring and non-naturally occurring. Furthermore, antibodies include chimeric antibodies, wholly synthetic antibodies, single chain antibodies, and fragments thereof (e.g. Fab). Antibodies may be human or nonhuman. Nonhuman antibodies may be humanized by recombinant methods to reduce their immunogenicity in humans.

“Collectively,” with respect to the binding of a plurality of antibodies to a common antigen, means that all such antibodies bind to that antigen, even if one antibody recognizes a different epitope of the antigen than does another. Likewise, if a plurality of antibodies collectively bind to a plurality of antigens, every such antigen is recognized by at least one such antibody. For example, a plurality of anti-IGFBP-3 antibodies collectively bind to both intact IGFBP-3 and degraded IGFBP-3 each if intact IGFBP-3 and degraded IGFBP-3 is recognized by at least one such antibody.

“Colon cancer” shall mean a cancerous malignancy that arises from the inner lining of the colon.

“Degraded IGFBP-3” shall mean IGFBP-3 which is not biologically active (e.g. arising from proteolytic degradation of intact IGFBP-3). “Degraded IGFBP-3” and “IGFBP-3 fragment” are used synonymously.

“Detectably-labeled” antibody includes, without limitation, an antibody having a detectable substance physically bound thereto either covalently or noncovalently. The label bound to the antibody can be detected directly (e.g. if the label is a fluorescent or radioactive substance) or indirectly (e.g. if the label is biotin whose detection is performed using fluorescently-labeled streptavidin).

“Increased risk” for developing colon cancer with respect to a human subject afflicted with long-lasting IBD means a probability for that subject's developing colon cancer which is greater than the median probability for developing colon cancer among human subjects afflicted with long-lasting IBD.

“Intact IGFBP-3” shall mean IGFPB-3 that is biologically active.

“Indicative of an increased risk of developing colon cancer,” with respect to the concentration of intact IGFBP-3 in a cell-free bodily fluid sample, includes, without limitation, a concentration of from 0 to 500 ng of intact IGFBP-3 per ml of undiluted suitable cell-free bodily fluid sample. By contrast, a concentration of from 800 to 2000 ng of intact IGFBP-3 per ml of undiluted suitable cell-free bodily fluid sample would not be indicative of an increased risk of colon cancer. In one embodiment, each of the concentrations of from 0 ng-100 ng, 100 ng-200 ng, 200 ng-300 ng, 300 ng-400 ng, and 400 ng-500 ng of intact IGFBP-3 per ml of undiluted cell-free bodily fluid sample is indicative of an increased risk of developing colon cancer.

“Long-lasting” IBD shall mean IBD which has persisted for about five years or more.

“Operably affixed,” with respect to a plurality of antibodies being affixed to a solid substrate, shall mean that at least a portion of affixed antibodies are oriented so that they can bind their respective antigens.

“Suitable cell-free bodily fluid sample” includes, without limitation, blood serum, blood plasma, saliva, cerebrospinal fluid, and synovial fluid. Samples of cell-free bodily fluid can remain undiluted, or be diluted, while the instant methods are performed. However, as is clear herein, intact IGFBP-3 concentrations indicating increased risk of colon cancer (e.g. 0-500 ng/ml) refer to concentrations of IGFBP-3 present in the undiluted cell-free bodily fluid.

“Total IGFBP-3” shall mean the sum of intact IGFBP-3 and degraded IGFBP-3.

### EMBODIMENTS OF THE INVENTION

The subject invention provides a method for determining whether a human subject afflicted with long-lasting irritable bowel disease (IBD) has an increased risk for developing colon cancer comprising:
(a) determining the concentration of intact IGFBP-3 in a suitable cell-free bodily fluid sample taken from the subject; and
(b) determining whether the concentration of intact IGFBP-3 determined in step (a) is indicative of an increased risk of colon cancer in a human subject afflicted with long-lasting IBD.

In one embodiment, the subject has Crohn's disease. In another embodiment, the subject has ulcerative colitis. In a further embodiment, the subject has had IBD for five years or more, seven years or more, eight years or more, or 20 years or more.

In another embodiment, in step (b), the concentration of intact IGFBP-3 indicative of an increased risk of colon cancer in a human subject afflicted with long-lasting IBD is a concentration of from 0 to 500 ng of intact IGFBP-3 per ml of undiluted suitable cell-free bodily fluid sample.

In yet another embodiment, determining the concentration of intact IGFBP-3 in the sample comprises the steps of (a) determining the total amount of IGFBP-3 in an aliquot of the sample and (b) determining the percentage of total IGFBP-3 which constitutes intact IGFBP-3, wherein (a) and (b) can be performed concurrently or sequentially in any order. In one embodiment, step (a) is performed using an ELISA assay which permits the quantification of total IGFBP-3 and step (b) is performed using a Western blot which permits determining the relative amounts of intact and degraded IGFBP-3 which, together, constitute total IGFBP-3.

In one embodiment, the suitable cell-free bodily fluid sample is blood serum, blood plasma, saliva, cerebrospinal fluid, or synovial fluid. In the preferred embodiment, the sample is blood serum or blood plasma.

This invention also provides a kit for determining whether a human subject afflicted with long-lasting irritable bowel disease (IBD) has an increased risk for developing colon cancer comprising, in separate compartments:
(a) an ELISA plate having operably affixed thereto anti-IGFBP-3 antibodies, wherein collectively the antibodies bind to both intact and degraded IGFBP-3;
(b) magnetic beads having operably affixed thereto anti-IGFBP-3 antibodies, wherein collectively the antibodies bind to both intact and degraded IGFBP-3; and
(c) a detectably-labeled antibody which binds to both intact and degraded IGFBP-3, wherein the detectably-labeled antibody permits the quantification of total IGFBP-3 bound to the ELISA plate of (a).

In one embodiment, the antibodies of (a) and (b) are polyclonal antibodies. In another embodiment, the kit further comprises instructions for use.

This invention is illustrated in the Experimental Details section which follows. This section is set forth to aid in an understanding of the invention but is not intended to, and should not be construed to, limit in any way the invention as set forth in the claims which follow thereafter.

### EXPERIMENTAL DETAILS

### Synopsis

IGFBP-3, originally described as a protein that binds and limits availability of the major cell growth factor, IGF-I (insulin-like growth factor I). It is known now that IGFBP-3 has its own IGF-I-independent tumor growth suppressive properties. Several studies have demonstrated that low serum IGFBP-3 levels, when adjusted for IGF-I concentration, represent a risk factor for the developing of several types of cancer, including colon cancer. These studies measured the level of total IGFBP-3, which is a sum of an intact bioactive protein and its degradation products. IGFBP-3, a 43-45 kDa doublet protein is a component of human plasma and can be detected as an intact bioactive protein and its inactive degradation products. The concentration of IGFBP-3 in tumor-free patients is relatively high, 6650±1550 ng/ml measured in ELISA as total protein and 2961±1118 ng/ml of intact bioactive protein assessed in a combined ELISA and Western Blot assay. Patients with active inflammatory bowel disease develop a moderate decline in intact IGFBP-3, 2119±686 ng/ml in UC and 1536±1186 ng/ml in CD. These levels normalize during the remission, 4258±1864 ng/ml in UC and 3043±1888 ng/ml in CD. A dramatic depletion of intact IGFBP-3 occurred in 14.3% of patients with active moderate-to-severe IBD and in 4-4.8% of patients with mild IBD and in remission. In total, it was observed in 7.4% of patients. Interestingly, 7-8% of patients develop colon cancer after 20 years of IBD history. This invention uses a combined ELISA and Western Blot IGFBP-3 assay to identify and screen a category of IBD patients at risk for developing colon cancer, who may benefit from IGFBP-3 replacement therapy.

### A. METHODS

Patients: Patients with documented UC and CD according to international criteria (18,19), who attended the regional outpatient specialized IBD clinic were eligible for inclusion in this study. A total of 132 consenting patients comprise the IBD population of this study. Seventy seven of the patients carried the diagnosis of UC of which 42 were males with a median age of 45 years (range 18-87) and 35 were females with a median age of 41 years (range 18-68). A total of 55 CD patients were also included: 29 were females with a median age of 39 years (20-69) and 26 were males with a median age of 36 years (range 20-69). The control group consisted of 16 patients with diverticular disease, 10 males and 6 females with a median age of 51 years (39-63). None of the control patients had been taking anti-inflammatory drugs in the preceding 2 weeks, including aspirin and NSAIDs. The UC patients were all receiving maintenance treatment with mesalamine 2.4-4.8 g daily, and none had received glucocorticosteroids or other immunosuppressive drugs, including azathioprine/6-mercaptopurine.

Clinical data regarding each patient's condition were obtained and recorded. The information included the following 1) number of bowel movements daily, 2) the presence of blood, mucus, and pus in the stools, 3) abdominal pain or discomfort, 4) fever and 5) other extra intestinal manifestations. The clinical activity of inflammation was assessed using a semi quantitative scale (0=remission; 1=slight activity; 2=moderate activity; 3=severe activity (the latter two were combined)) (20).

Plasma samples: Blood was drawn from an antecubital vein in EDTA containing tubes (final concentration 1.8 mg/ml) and mixed gently. Following this procedure the blood was centrifuged (10 min, 1600 g) at ambient temperature. Plasma was isolated and stored at −80° C. until the analysis was performed.

IGFBP-3 ELISA

The concentration of total IGFBP-3 was assessed using the ELISA kit (Diagnostic Systems Laboratories Inc., Webster, TX) according to the manufacturer's instructions and a microplate reader, EL×800 (Bio-Tec, Virginia Beach, Va.). Plasma samples were applied in duplicates in 1:100 dilutions. The sensitivity of the assay was 1 ng/ml.

IGFBP3 Western Blot Analysis Assisted by Immunomagnetic Separation

Total IGFBP-3 protein was immunomagentically separated from the plasma samples. Briefly, EDTA plasma samples (300 μl) were incubated with magnetic beads (Dynal Biotech, Oslo, Norway) coated with the anti-IGFBP3 antibody (R&D Systems, Minneapolis, Minn.). The products were isolated using a magnet and electrophoretically separated on 18% SDS-polyacrilamide gels under reducing conditions. Proteins were transferred to a nitrocellulose membrane. After being blocked with 6% milk, the membrane was incubated with a polyclonal mouse antibody to human IGFBP-3 (R&D Systems) and after several washes with peroxidase labelled antibody to mouse IgG (Pierce Biotechnology, Rockford, Ill.). After extensive washes, membranes were incubated with a chemiluminescent reagent (Pierce) and exposed to an X-ray film. The ratio of “intact IGFBP3/total IGFBP3” as well as the ratio of “IGFBP3 fragments/total IGFBP3” was determined electronically using Scion Image software. The concentration of intact IGFBP3 was calculated as follows: Intact IGFBP-3 (ng/ml)=Total IGFBP-3 (ng/ml) [ELISA derived concentration]×(intact IGFBP-3/total IGFBP-3) [ratio derived from Western Blot analysis].

Zymography

Serum samples (3 μl/sample) diluted with loading buffer were electrophoretically separated on gelatine zymogram pre-cast gels (Invitrogen, Carlsbad, Calif.). After separation, samples were renatured according to the manufacturer's instructions, stained with Coomassie Blue (Bio-Rad Laboratories, Hercules, Calif.) and counterstained with 0.1% methylene blue solution (LabChem, Pittsburgh, Pa.). The image was obtained by scanning.

MMP-9 ELISA

ELISA plates (Corning Incorporated, Corning, N.Y.) were coated with a monoclonal antibody to human MMP-9 (R&D Systems, Minneapolis, Minn.). After several washes the plates were blocked with a 3% milk solution and serial dilutions of human plasma were applied in duplicates with a starting dilution of 1:10. Subsequently, the plates were washed, and the incubations with polyclonal biotinylated antibodies to human MMP-9 (Bio-Rad) and streptavidin-peroxidase (BD Pharmingen, San Jose, Calif.). The reaction was developed with teteramethylbenzidine solution (Sigma Chemical, St. Louis, Mo.) and stopped with a sulphuric acid solution (Sigma). A recombinant human MMP-9 (R&D Systems) was used as a standard. The reaction was evaluated using EL×800 microplate reader (Bio-Tek Instruments, Inc, Winooski, Vt.).

Ethics

This investigation was approved by the Scientific Ethics Committee of the Copenhagen County (KA 03036) and by Western Institutional Review Board (Olympia, Wash., USA, 2432WIRB) and performed in accordance with the Helsinki V Declaration.

Statistical Analysis

The results are presented as medians and (range values) and analyzed using non-parametric statistical tests; unpaired data were tested by the Mann-Whitney rank sum test and paired data by Wilcoxon's test. A P value of 0.05 or less was considered statistically significant.

### B. RESULTS

Total IGFBP-3

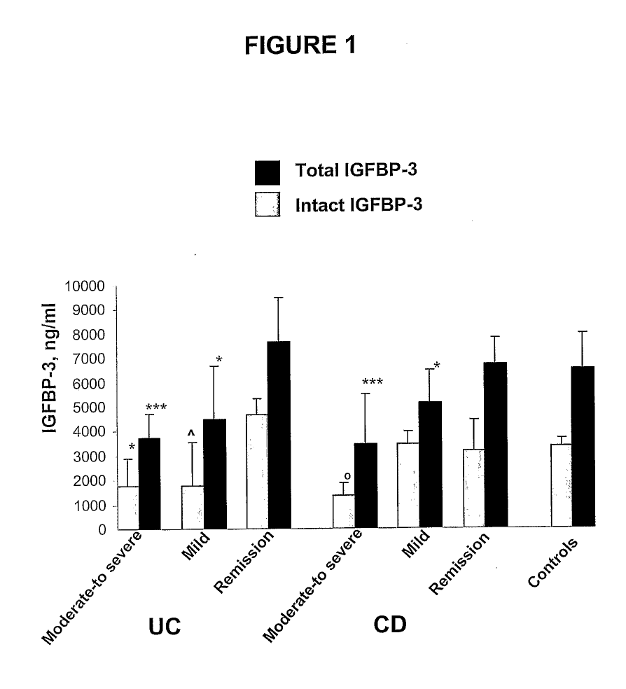

Total IGFBP-3 Levels were measured in ELISA in plasma from IBD patients and controls. The median total IGFBP-3 concentration was significantly lower in patients with active moderate-to-severe UC, 3760 (2786-5234) ng/ml and in patients with active moderate-to-severe CD, 3448 (2954-5537) ng/ml compared to controls, 6520 (3414-8861) ng/ml (p<0.005 for both comparisons) or to the group of patients with inactive disease, UC 7675 (2597-11941) ng/ml (p<0.001) and CD 6711 (3146-12146) ng/ml (p<0.005) (FIG. 1). The median level of total IGFBP-3 in patients with mild UC, 4480 (1551-11754) ng/ml and mild CD, 5163 (1227-7797) ng/ml was moderately, albeit significantly lower than in patients in remission or controls (p<0.05) (FIG. 1). As mentioned, the IGFBP-3 ELISA detects both the intact protein and its fragments. Because cell growth regulatory activity pertains only to the intact IGFB-3 molecule, we further determined the concentration of intact IGFBP-3.

Concentration of Intact IGFBP-3

Patients from 3 groups, active moderate-to severe (n=14), mild (n=24) and remission (n=21) were randomly selected to analyze the concentration of intact IGFBP-3 in combined ELISA and Western Blot analysis. The median intact IGFBP-3 concentration was significantly decreased in patients with active moderate-to-severe UC, 1760 (1451-3172) ng/ml when compared to the median concentration in inactive UC, 4676 (1048-7228) ng/ml or in controls, 3360 (1115-4565) ng/ml (p<0.05). Similarly, the median intact IGFBP-3 level in patients with active moderate-to-severe CD 1378 (371-3904) ng/ml was significantly lower than in controls (p<0.05) and tended to be lower than in patients with remission, 3169 (197-8376) ng/ml (p=0.09) (FIG. 1). A significant decline in the level of intact IGFBP-3 was also found in UC (but not CD) patients with active mild disease 1763 (457-5508) ng/ml versus UC remission 4676 (1048-7228) ng/ml (p<0.05).

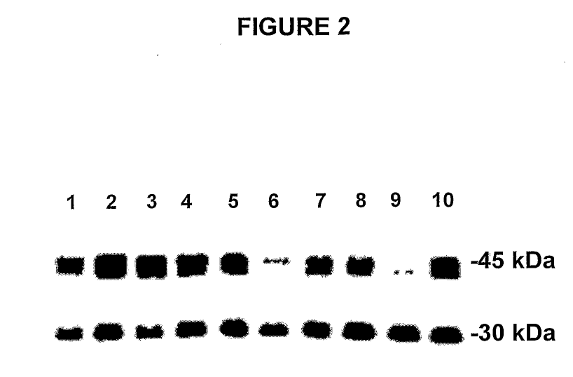

Interestingly, a dramatic depletion of intact IGFBP-3 (FIG. 2) was found in 14.3% of patients with active moderate to severe IBD (combined UC and CD), 4% of patients with active mild IBD and 4.8% of patients with inactive IBD. In total, this effect was observed in 7.4% of IBD. We further assessed the serum proteases that are known to degrade IGFBP-3.

Zymography

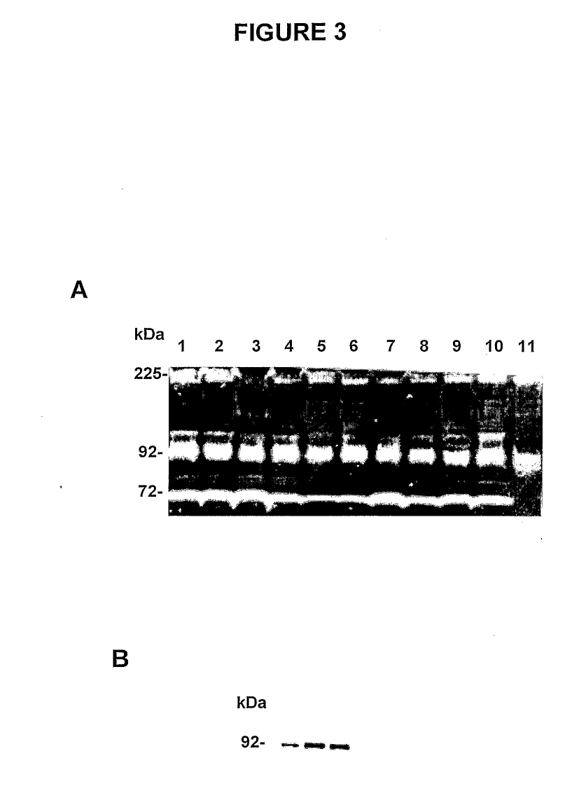

Serum proteolytic activity was assessed in zymography. The most abundant serum protease had a molecular size consistent with MMP-9 pro form (FIG. 3); the nature of this protein was further confirmed in Western Blot analysis to be MMP-9 (FIG. 3). We further assessed the plasma concentration of MMP-9 pro-form in ELISA in patients with IBD. The median concentration of MMP-9 in patients with moderate-to-severe IBD, 56 (16-999) ng/ml was comparable to the results found in patients with mild IBD, 69 (21-506) ng/ml, patients in remission, 92 (40-209) ng/ml and in controls, 114 (2-436) ng/ml.

### C. DISCUSSION

We found a significant decrease in the level of total IGFBP-3 in IBD patients with moderate to severe active disease compared to IBD patients in remission and controls. Smaller, yet still significant reduction in total IGFBP-3 concentration was noted in patients with mildly active IBD when compared to the same controls. These results confirm previously reported studies concerning IGFBP-3 levels in the setting of IBD (7-9). We further assessed the level of bioactive intact IGFBP-3 in the same groups and found that in the majority of patients intact protein concentration was decreased to a similar extent as observed with the total protein concentration. We show here for the first time that patients with active moderate-to-severe IBD have a significant reduction in the level of bioactive IGFBP-3. This observation supports the hypothesis that an increase in epithelial cell proliferation rate (1-3) in IBD patients with moderate to severe disease activity may be linked to a diminished concentration of the growth regulatory factor, IGFBP-3.

A decline in IGFBP-3 concentration in active IBD may be linked either to its decreased production or an increased degradation. It is difficult to directly evaluate IGFBP-3 production in humans since it is not clear which tissues are responsible for its generation. In IGFBP-3 transgenic mice, the major sites of IGFBP-3 expression are kidneys and lungs (21). We indirectly assessed production by determining the concentration of intact IGFBP-3 in active disease versus remission versus controls and then comparing to the concentration of total IGFBP-3 for the same groups. It is apparent from our studies that the extent of the decrease in the level of intact IGFBP-3 is proportional to observed decrease in total IGFBP-3 in active moderate-to-severe IBD. These results suggest that a reduced IGFBP-3 production, rather than increased proteolysis, accounts for the observed decrease in the concentration of this protein in plasma. It is possible that proteases are released in an inactive, pro-form in active IBD. In this case, protease activation and subsequent IGFBP-3 degradation may occur locally in tissue on cell plasma membranes (22). In an effort to assess this hypothesis, plasma from IBD patients was screened for gelatinases; the most abundant proteolytic band was consistent with the pro-form of MMP-9. However, when studied, the median MMP-9 concentration was not significantly different between the groups.

Although the majority of IBD patients with active disease demonstrated moderate decreases in intact IGFBP-3 levels, dramatic depletion of intact IGFBP-3 was noted in 14.3% of patients with moderate to severe disease activity. This depletion occurred also in 4-4.8% of patients with mildly active IBD or in remission. In total, striking decreases in the level of intact IGFBP-3 were observed in 7.4% of IBD patients. Interestingly, in regard to patients who have had IBD for more than 20 years, about 7-8% of patients will develop colon cancer (23). It has been noted, in the general population, that after controlling for IGF-I levels, lower IGFBP-3 concentration is associated with a greater risk of colon cancer development (24). This invention is based on the notion that the observed significant decrease in the concentration of intact IGFBP-3 is a risk factor for the development of colon cancer in IBD patients.

We conclude that the level of bioactive, i.e. intact IGFBP-3, is moderately, yet significantly decreased in patients with active moderate or severe IBD, and a striking depletion of intact IGFBP-3 is observed in 7.4% of patients. The decrease in IGFBP-3 concentration is not associated with altered levels of an IGFBP-3 degrading enzyme, MMP-9.

### REFERENCES

1. Serafini E P, Kirk A P, Chambers T J. Rate and pattern of epithelial cell proliferation in ulcerative colitis. Gut 22: 648-652, 1981.
2. Noffsinger A E, Miller M A, Cusi M V, et al. The pattern of cell proliferation in neoplastic and nonneoplastic lesions of ulcerative colitis. Cancer 78:2307-12, 1996.
3. Shinozaki M, Watanabe T, Kubota Y, et al. High proliferative activity is associated with dysplasia in ulcerative colitis. Dis Colon Rectum 43(10 Suppl):S34-9, 2000.
4. Di Sabatino A, Ciccocioppo R, Luinetti O, et al. Increased enterocyte apoptosis in inflamed areas of Crohn's disease. Dis Colon Rectum 46:1498-507, 2003.
5. Yukawa M, Iizuka M, Horie Y, et al. Systemic and local evidence of increased Fas-mediated apoptosis in ulcerative colitis. Int J Colorectal Dis 17:70-6, 2002.
6. Strater J, Wellisch I, Riedl S, et al. Related CD95 (APO-1/Fas)-mediated apoptosis in colon epithelial cells: a possible role in ulcerative colitis. Gastroenterology 113:160-7, 1997.
7. Akobeng A I, Clayton P E, Miller V, et al. Low serum concentrations of insulin-like growth factor-I in children with active Crohn disease: effect of enteral nutritional support and glutamine supplementation. Scand J Gastroenterol 37:1422-7, 2002.
8. Gronbek H, Thogersen T, Frystyk J, et al. Low free and total insulinlike growth factor I (IGF-I) and IGF binding protein-3 levels in chronic inflammatory bowel disease: partial normalization during prednisolone treatment. Am J Gastroenterol 97:673-8, 2002.
9. Katsanos K H, Tsatsoulis A, Christodoulou D, et al. Reduced serum insulin-like growth factor-1 (IGF-1) and IGF-binding protein-3 levels in adults with inflammatory bowel disease. Growth Horm IGF Res 11:364-7, 2001.
10. Lee K W, Liu B, Ma L, et al. Cellular internalization of insulin-like growth factor binding protein-3: distinct endocytic pathways facilitate re-uptake and nuclear localization. J Biol Chem 279:469-76, 2004.
11. Schedlich L J, Le Page S L, Firth S M, et al. Nuclear import of insulin-like growth factor-binding protein-3 and −5 is mediated by the importin beta subunit. J Biol Chem 275:23462-70, 2000.
12. Schedlich L J, Young T F, Firth S M, et al. Insulin-like growth factor-binding protein (IGFBP)-3 and IGFBP-5 share a common nuclear transport pathway in T47D human breast carcinoma cells. J Biol Chem 273:18347-52, 1998.
13. Butt A J, Firth S M, King M A, et al. Insulin-like growth factor-binding protein-3 modulates expression of Bax and Bcl-2 and potentiates p53-independent radiation-induced apoptosis in human breast cancer cells. J Biol Chem 275:39174-81, 2000.
14. Gill Z P, Perks C M, Newcomb P V, et al. Insulin-like growth factor-binding protein (IGFBP-3) predisposes breast cancer cells to programmed cell death in a non-IGF-dependent manner. J Biol Chem 272:25602-7, 1997.
15. Hong J, Zhang G, Dong F, et al. Insulin-like growth factor (IGF)-binding protein-3 mutants that do not bind IGF-I or IGF-II stimulate apoptosis in human prostate cancer cells. J Biol Chem 277:10489-97, 2002.
16. Lee H Y, Chun K H, Liu B, et al. Insulin-like growth factor binding protein-3 inhibits the growth of non-small cell lung cancer. Cancer Res 62:3530-7, 2002.
17. Kirman I, Cekic V, Poltaratskaia N, et al. Plasma from patients undergoing major open surgery stimulates in vitro tumor growth: Lower insulin-like growth factor binding protein 3 levels may, in part, account for this change. Surgery 132:186-92, 2002.
18. Langholz E, Munkholm P, Nielsen O H, et al. Incidence and prevalence of ulcerative colitis in Copenhagen County from 1962 to 1987. Scand J Gastroenterol 26: 1247-56, 1991.
19. Munkholm P, Langholz E, Nielsen OH, et al. Incidence and prevalence of Crohn's disease in the county of Copenhagen, 1962 -1987: A sixfold increase in incidence. Scand J Gastroenterol 27: 609-14, 1992.
20. Tvede M, Bondesen S, Nielsen O H, et al. Serum antibodies to Bacteroides species in chronic inflammatory bowel disease. Scand J Gastroenterol 18: 403-409, 1983.
21. Modric T, Silha J V, Shi Z, et al. Phenotypic manifestations of insulin-like growth factor-binding protein-3 overexpression in transgenic mice. Endocrinology 142:1958-67, 2001.
22. Toth M, Chvyrkova I, Bernardo M M, et al. Pro-MMP-9 activation by the MT1-MMP/MMP-2 axis and MMP-3: role of TIMP-2 and plasma membranes. Biochem Biophys Res Commun 308:386-95, 2003.
23. Greenstein A J. Cancer in inflammatory bowel disease. Mt Sinai J Med 67:227-40, 2000.
24. Ma J, Pollak M N, Giovannucci E, et al. Prospective study of colorectal cancer risk in men and plasma levels of insulin-like growth factor (IGF)-I and IGF-binding protein-3. J Natl Cancer Inst 91:620-5, 1999.

What is claimed is:

1. A method for determining whether a human subject afflicted with long-lasting irritable bowel disease (IBD) has an increased risk for developing colon cancer comprising:
(a) determining the concentration of intact IGFBP-3 in a suitable cell-free bodily fluid sample taken from the subject; and
(b) determining whether the concentration of intact IGFBP-3 determined in step (a) is indicative of an increased risk of colon cancer in a human subject afflicted with long-lasting IBD.

2. The method of claim 1, wherein the subject has Crohn's disease.

3. The method of claim 1, wherein the subject has ulcerative colitis.

4. The method of claim 1, wherein the subject has had IBD for five years or more.

5. The method of claim 4, wherein the subject has had IBD for seven years or more.

6. The method of claim 5, wherein the subject has had IBD for eight years or more.

7. The method of claim 6, wherein the subject has had IBD for 20 years or more.

8. The method of claim 1, wherein in step (b), the concentration of intact IGFBP-3 indicative of an increased risk of colon cancer in a human subject afflicted with long-lasting IBD is a concentration of from 0 to 500 ng of intact IGFBP-3 per ml of undiluted suitable cell-free bodily fluid sample.

9. The method of claim 1, wherein determining the concentration of intact IGFBP-3 in the sample comprises the steps of (a) determining the total amount of IGFBP-3 in an aliquot of the sample and (b) determining the percentage of total IGFBP-3 which constitutes intact IGFBP-3, wherein (a) and (b) can be performed concurrently or sequentially in any order.

10. The method of claim 9, wherein step (a) is performed using an ELISA assay which permits the quantification of total IGFBP-3 and step (b) is performed using a Western blot which permits determining the relative amounts of intact and degraded IGFBP-3 which, together, constitute total IGFBP-3.

11. The method of claim 1, wherein the suitable cell-free bodily fluid sample is blood serum, blood plasma, saliva, cerebrospinal fluid, or synovial fluid.

12. The method of claim 11, wherein the suitable cell-free bodily fluid sample is blood serum.

13. The method of claim 11, wherein the suitable cell-free bodily fluid sample is blood plasma.

14. A kit for determining whether a human subject afflicted with long-lasting irritable bowel disease (IBD) has an increased risk for developing colon cancer comprising, in separate compartments:
(a) an ELISA plate having operably affixed thereto anti-IGFBP-3 antibodies, wherein collectively the antibodies bind to both intact and degraded IGFBP-3;
(b) magnetic beads having operably affixed thereto anti-IGFBP-3 antibodies, wherein collectively the antibodies bind to both intact and degraded IGFBP-3; and
(c) a detectably-labeled antibody which binds to both intact and degraded IGFBP-3, wherein the detectably-labeled antibody permits the quantification of total IGFBP-3 bound to the ELISA plate of (a).

15. The kit of claim 14, wherein the antibodies of (a) and (b) are polyclonal antibodies.

16. The kit of claim 14, further comprising instructions for use.
